(19) United States
(12) Patent Application Publication
(10) Pub. No.: US 2026/0294538 A1
(43) Pub. Date: Oct. 1, 2026

(54) URINARY STONE EDGE DETECTION FOR LASER LITHOTRIPSY

(71) Applicant: Lumenis Ltd., Yokneam Ilit (IL)

(72) Inventor: Majd Sleem, Natzrat Ilit (IL)

(73) Assignee: Lumenis Ltd., Yokneam Ilit (IL)

(21) Appl. No.: 19/634,260

(22) Filed: Mar. 31, 2026

Related U.S. Application Data
(60) Provisional application No. 63/781,055, filed on Mar. 31, 2025.

(51) Int. Cl. A61B 18/26; A61B 18/00; A61B 18/20

(52) U.S. Cl. CPC A61B 18/26; A61B 2018/00505; A61B 2018/00642; A61B 2018/00678; A61B 2018/00708; A61B 2018/00773; A61B 2018/2035

(57) ABSTRACT

Described herein are laser emitting medical devices and techniques implementable by laser emitting medical devices to distinguish between targeted and non-targeted objects based on fluorescence reflected from an object responsive to incidence of laser beam on the object. The disclosure provides to determine an average of the fluorescence over a number of samples and to determine whether the object is a targeted or non-targeted object based on the average.

This application claims the benefit of priority under 35 USC § 119 to United States Provisional Patent Application Serial No. 63/781,055, filed Mar. 31, 2025, which is incorporated by reference herein in its entirety and for all purposes.

### TECHNICAL FIELD

The present disclosure generally relates to surgical laser system. Particularly, but not exclusively, the present disclosure relates to identifying an edge of a urinary stone during a laser lithotripsy procedure.

### BACKGROUND

Medical lasers are used in a variety of procedures. Among a number of such procedures, laser energy is directed towards a target using an optical fiber as a conduit for the laser energy. One such procedure, to address renal calculi (e.g., kidney stones) is ureteral endoscopy, or lithotripsy. A typical lithotripsy procedure involves inserting an endoscopic (e.g., ureteroscope, or the like) into the urinary tract of a patient. A camera of the endoscope is used to locate the calculi for removal. As part of the removal process, an optical fiber is inserted into the treatment area via a working channel of the endoscope. Laser energy is generated by a surgical laser console and the target is illuminated and/or irradiated with the laser energy via the optical fiber. The laser energy operates to disintegrate, dust, ablate, fragment and/or destroy the calculi.

As will be appreciated by those of ordinary skill in the art, it is important to accurately target the renal calculi and avoid targeting tissue adjacent to the renal calculi. Thus, there is a need to identify when the optical fiber is “aimed” at renal calculi and when it is not.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides a surgical laser console configured to determine whether renal calculi (generally referred to herein as a stone) or the edge renal calculi is being targeted. For example, a surgical laser console of the present disclosure can be configured to distinguish between a stone and tissue and to enable and/or disable lasing based on determining whether stone or tissue is being targeted by an optical fiber coupled to the surgical laser console. Further, the present disclosure provides that the surgical laser console can be configured to determine whether an edge of the stone is being targeted by the optical fiber.

Some embodiments of the disclosure can be implemented as a computer implemented method. The method can comprise receiving, at a processor, electrical signals generated by a light detector, the electrical signals comprising indications of a fluorescence of reflected light received at the light detector; deriving an average of the fluorescence over the last N samples of the electrical signals; determining whether the average of the fluorescence over the last N samples is greater than or greater than or equal to a threshold value; and sending, responsive to the determination, a control signal to a therapeutic laser source.

In some embodiments of the method, sending the control signal to the therapeutic laser source comprises sending a first control signal to the therapeutic laser source based on a determination that the average of the fluorescence over the last N samples is greater than or greater than or equal to the threshold value; or sending a second control signal to the therapeutic laser source based on a determination that the average of the fluorescence over the last N samples is not greater than or not greater than or equal to the threshold value, wherein the first control signal causes the therapeutic laser source to generate a therapeutic laser beam, and wherein the second control signal causes the therapeutic laser source to not generate the therapeutic laser beam.

In some embodiments, the method can comprise deriving an average of the fluorescence over the last M samples of the electrical signals, where M is different than N; and determining whether the average of the fluorescence over the last M samples is greater than the threshold value, wherein sending the control signal to the therapeutic laser source is responsive to both determinations.

In some embodiments of the method, sending the control signal to the therapeutic laser source comprises sending a first control signal to the therapeutic laser source based on a determination that the average of the fluorescence over the last N samples and the average fluorescence over the last M samples is greater than or greater than or equal to the threshold value; or sending a second control signal to the therapeutic laser source based on a determination that the average of the fluorescence over the last N samples or the average fluorescence over the last M samples is not greater than or not greater than or equal to the threshold value, wherein the first control signal causes the therapeutic laser source to generate a therapeutic laser beam, and wherein the second control signal causes the therapeutic laser source to not generate the therapeutic laser beam.

In some embodiments of the method, N is less than M.

In some embodiments of the method, N is less than 32 and M is greater than 32.

In some embodiments of the method, N is between 3 and 31 and M is between 32 and 96.

In some embodiments, the method can comprise generating, by an interrogation sub-system during a surgical laser procedure, an interrogation laser beam; and coupling the interrogation laser beam to an optical fiber, wherein the optical fiber is disposed in a treatment environment, and wherein the reflected light is light reflected from an object in the treatment environment responsive to incidence of the interrogation laser beam on the object.

In some embodiments of the method, the object is a urinary stone.

Some embodiments of the disclosure can be implemented as a surgical laser system. The surgical laser system can comprise a therapeutic laser source configured to generate a therapeutic laser beam; an interrogation sub-system comprising an interrogation laser source configured to generate an interrogation laser beam, a light detector, and a beam splitter; optics configured to condition the therapeutic laser beam and the interrogation laser beam and couple the therapeutic laser beam and the interrogation laser beam to an optical fiber; a processor coupled to the light detector; and memory coupled to the processor, the memory comprising instructions that when executed cause the processor to receive, from the light detector, electrical signals comprising indications of a fluorescence of reflected light, derive an average of the fluorescence over the last N samples of the electrical signals, determine whether the average of the fluorescence over the last N samples is greater than or greater than or equal to a threshold value, and send, responsive to the determination, a control signal to the therapeutic laser source, wherein the reflected light is light reflected from an object responsive to incidence of the interrogation laser beam on the object.

In some embodiments of the surgical laser system, the instructions when executed by the processor further cause the processor to send a first control signal to the therapeutic laser source based on a determination that the average of the fluorescence over the last N samples is greater than or greater than or equal to the threshold value; or send a second control signal to the therapeutic laser source based on a determination that the average of the fluorescence over the last N samples is not greater than or not greater than or equal to the threshold value, wherein the first control signal causes the therapeutic laser source to generate a therapeutic laser beam, and wherein the second control signal causes the therapeutic laser source to not generate the therapeutic laser beam.

In some embodiments of the surgical laser system, the instructions when executed by the processor further cause the processor to derive an average of the fluorescence over the last M samples of the electrical signals, where M is different than N; and determine whether the average of the fluorescence over the last M samples is greater than the threshold value, wherein sending the control signal to the therapeutic laser source is responsive to both determinations.

In some embodiments of the surgical laser system, the instructions when executed by the processor further cause the processor to send a first control signal to the therapeutic laser source based on a determination that the average of the fluorescence over the last N samples and the average fluorescence over the last M samples is greater than or greater than or equal to the threshold value; or send a second control signal to the therapeutic laser source based on a determination that the average of the fluorescence over the last N samples or the average fluorescence over the last M samples is not greater than or not greater than or equal to the threshold value, wherein the first control signal causes the therapeutic laser source to generate a therapeutic laser beam, and wherein the second control signal causes the therapeutic laser source to not generate the therapeutic laser beam.

In some embodiments of the surgical laser system, N is 6 and M is 64.

In some embodiments of the surgical laser system, the object is a urinary stone.

Some embodiments of the disclosure can be implemented as at least one machine readable storage devices storing instructions that when executed by a processor of a surgical laser system cause the surgical laser system to receive, from a light detector, electrical signals comprising indications of a fluorescence of reflected light, derive an average of the fluorescence over the last N samples of the electrical signals, determine whether the average of the fluorescence over the last N samples is greater than or greater than or equal to a threshold value, and send, responsive to the determination, a control signal to a therapeutic laser source of the surgical laser system, wherein the reflected light is light reflected from an object responsive to incidence of an interrogation laser beam, generated by the surgical laser system, on the object.

In some embodiments of the at least one machine readable storage devices, the instructions when executed by the processor further cause the surgical laser system to send a first control signal to the therapeutic laser source based on a determination that the average of the fluorescence over the last N samples is greater than or greater than or equal to the threshold value; or send a second control signal to the therapeutic laser source based on a determination that the average of the fluorescence over the last N samples is not greater than or not greater than or equal to the threshold value, wherein the first control signal causes the therapeutic laser source to generate a therapeutic laser beam, and wherein the second control signal causes the therapeutic laser source to not generate the therapeutic laser beam.

In some embodiments of the at least one machine readable storage devices, the instructions when executed by the processor further cause the surgical laser system to derive an average of the fluorescence over the last M samples of the electrical signals, where M is different than N; and determine whether the average of the fluorescence over the last M samples is greater than the threshold value, wherein sending the control signal to the therapeutic laser source is responsive to both determinations.

In some embodiments of the at least one machine readable storage devices, the instructions when executed by the processor further cause the surgical laser system to send a first control signal to the therapeutic laser source based on a determination that the average of the fluorescence over the last N samples and the average fluorescence over the last M samples is greater than or greater than or equal to the threshold value; or send a second control signal to the therapeutic laser source based on a determination that the average of the fluorescence over the last N samples or the average fluorescence over the last M samples is not greater than or not greater than or equal to the threshold value, wherein the first control signal causes the therapeutic laser source to generate a therapeutic laser beam, and wherein the second control signal causes the therapeutic laser source to not generate the therapeutic laser beam.

In some embodiments of the at least one machine readable storage devices, N is 6 and M is 64.

In some embodiments of the at least one machine readable storage devices, the object is a urinary stone.

### BRIEF DESCRIPTION OF THE FIGURES

To easily identify the discussion of any element or act, the most significant digit or digits in a reference number refer to the figure number in which that element is first introduced.

FIG. 1 illustrates a computer system in communication with a laser system. The laser system emitting laser light through an optical fiber and receiving reflected laser light through the optical fiber.

FIGS. 2A, 2B, and 2C illustrate an optic fiber emitting laser light in a variety of orientations with respect to a target.

FIG. 3 illustrates an example interrogation sub-system.

FIGS. 4, 5, and 6 respectively illustrate methods for distinguishing between a targeted and non-targeted object.

FIG. 7 illustrates a plot of fluorescence of urinary stones and tissue.

FIG. 8 illustrates computer executable instructions.

FIG. 9 illustrates a computing environment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

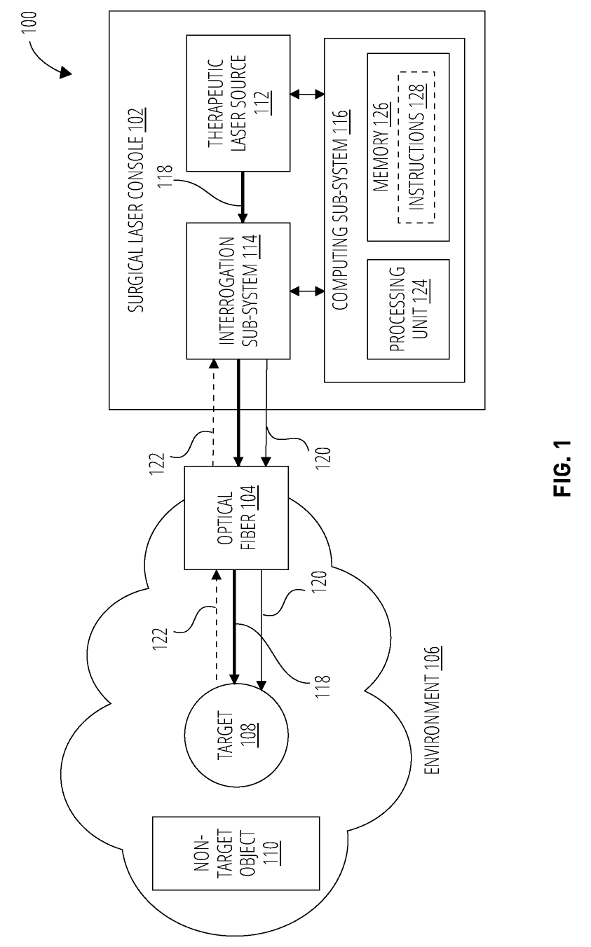

FIG. 1 shows an exemplary surgical laser system 100. In general, the surgical laser system 100 can be configured to be used in a laser lithotripsy procedure. During such a lithotripsy procedure, the surgical laser system 100 can be configured to distinguish between a stone and tissue and further to determine whether an edge of the stone is being targeted by an optical fiber.

The surgical laser system 100 comprises a surgical laser console 102 and an optical fiber 104 coupled to the surgical laser console 102. During a lithotripsy procedure, the optical fiber 104 can be inserted into an environment 106 in which a target 108 is to be treated. In an exemplary lithotripsy procedure, the target 108 is a stone (or renal calculi). However, it will be appreciated that the surgical laser console 102 and optical fiber 104 could be used to treat other types of targets (e.g., tissue, a tumor, a cyst, or the like). With illustrative examples, the target can be disposed in a liquid environment within a subject (e.g., a human, an animal, or the like). It is to be appreciated that the environment often includes non-target objects 110. For example, where the target is a urinary stone, the non-target object 110 will often be tissue.

Further, with some embodiments, the target 108 can be disposed in a liquid environment (e.g., urine, water, blood, etc.) For example, during a lithotripsy procedure, the target 108 may be a stone and the environment 106 can include a mixture of urine, irrigation fluid, and blood. Accordingly, the distal end 124 of the optical fiber 104 will also be disposed in the liquid environment during the procedure. When the therapeutic laser beam 118 are emitted from the optical fiber 104, some of the liquid may be vaporized and form a bubble or bubbles.

The surgical laser console 102 includes a therapeutic laser source 112, interrogation sub-system 114, and computing sub-system 116. During operation, the therapeutic laser source 112 is configured to generate therapeutic laser beam 118 while interrogation sub-system 114 is configured to generate interrogation laser beam 120.

In general, the therapeutic laser source 112 can be any of a variety of laser light sources (e.g., solid-state lasers, gas lasers, diode lasers, and fiber lasers). As a specific example, the therapeutic laser source 112 can be a Holmium (Ho) laser, such as a Ho:YAG laser. In yet another example, the therapeutic laser source 112 can be a Thulium (Tm) fiber laser. An example of the interrogation sub-system 114 is provided in greater detail below. However, in general the interrogation sub-system 114 is configured to generate interrogation laser beam 120, which can comprise multiple one or many laser beams, each having different wavelengths.

During operation, the therapeutic laser beam 118 is transmitted towards the target to “treat” the target. For example, where the target 108 is a urinary stone, the therapeutic laser beam 118 can be configured to fragment, dust, or otherwise destroy the target 108. Likewise, the interrogation laser beam 120 is transmitted through the optical fiber 104 and used to determine whether the optical fiber 104 is targeting, or pointed at, the target 108 or the non-target object 110. For example, during operation, the interrogation laser beam 120 can be transmitted into the environment 106 and light reflected from objects in the environment 106 (e.g., target 108, non-target object 110, etc.) can be received by the optical fiber 104 and transmitted back to the surgical laser console 102. This light is depicted as reflected light 122 is FIG. 1. The surgical laser console 102 can be configured to distinguish between the target 108 and the non-target object 110 based on the reflected light 122.

The computing sub-system 116 comprises at least a processing unit 124 and memory 126 storing instructions 128. With some examples, the computing sub-system 116 may be provided by a computing device (e.g., a laptop, a desktop, a mobile phone, a tablet, or the like). The processing unit 124 can include circuitry arranged to execute the instructions 128 stored in memory 126, which instructions when executed cause the computing sub-system 116 to send control signals to the therapeutic laser source 112 and the interrogation sub-system 114. For example, the processing unit 124 can execute instructions 128 to cause the computing sub-system 116 to send control signals to the interrogation sub-system 114 to cause the interrogation sub-system 114 to generate interrogation laser beam 120 and also to receive signals from the interrogation sub-system 114 comprising indications of reflected light 122 reflected from the target 108 or the non-target object 110 responsive to the interrogation laser beam 120.

Further, the processing unit 124 can execute instructions 128 to cause the computing sub-system 116 to determine whether the target 108 or the non-target object 110 is being targeted by the optical fiber 104 based on the reflected light 122. This is described in greater detail below. The processing unit 124 can execute instructions 128 to cause the computing sub-system 116 to send control signals to the therapeutic laser source 112 to cause the therapeutic laser source 112 to generate the therapeutic laser beam 118 based on a determination that the optical fiber 104 is targeting the target 108 and/or to cause the therapeutic laser source 112 to not generate the therapeutic laser beam 118 based on a determination that the optical fiber 104 is targeting the non-target object 110. Additionally, the processing unit 124 can execute instructions 128 to determine whether an edge of the target 108 is being targeted and to send control signals to the therapeutic laser source 112 based on the determination.

It is to be appreciated that the surgical laser console 102, and particularly the therapeutic laser source 112 and the interrogation sub-system 114 can include a variety of optical components besides the laser sources described above. For example, the therapeutic laser source 112 and the interrogation sub-system 114 can include (e.g., in different combinations as may be the case) light sources, polarizers, beam splitters, beam combiners, light detector, wavelength division multiplexers, collimators, circulators, etc.

Further, although not depicted, the surgical laser console 102 could include a display and/or other input and/or output devices configured to provide interaction with the surgical laser console 102 by a user (e.g., physician, or the like).

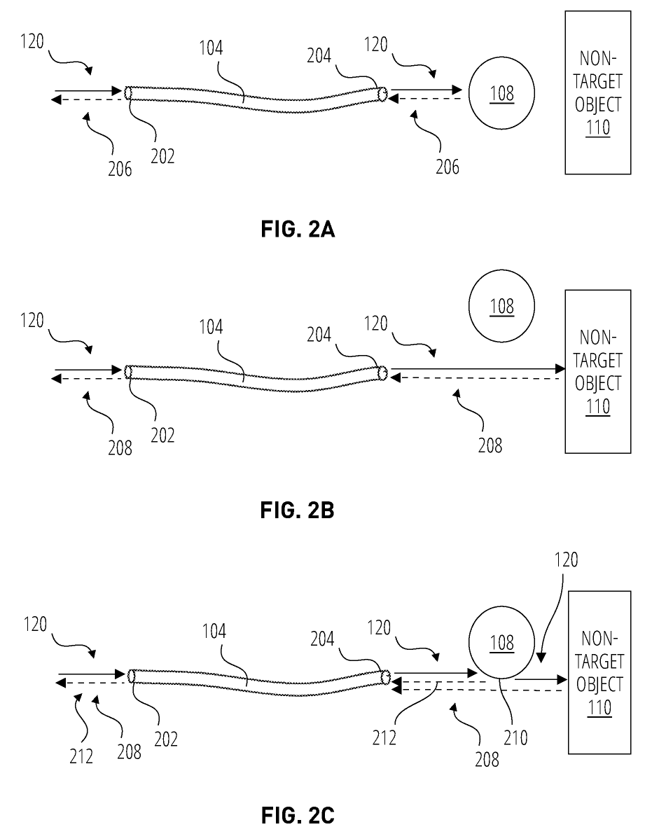

FIGS. 2A, 2B, and 2C depict examples of the optical fiber 104, target 108, and non-target object 110 of FIG. 1 in different orientations. For example, FIG. 2A depicts the optical fiber 104 targeting the target 108, FIG. 2B depicts the optical fiber 104 targeting the non-target object 110, and FIG. 2C depicts the optical fiber 104 targeting an edge of the target 108.

Turning specifically to FIG. 2A, the optical fiber 104 and particularly a proximal end 202 and a distal end 204 of the optical fiber 104 are depicted. The interrogation laser beam 120 can be coupled to the proximal end 202 of the optical fiber 104. The interrogation laser beam 120 can be transmitted through the optical fiber 104 and be emitted from the distal end 204 of the optical fiber 104.

As depicted in this figure, the optical fiber 104 is “targeting” the target 108. Said differently, the distal end 204 is pointed at the target 108 such that the interrogation laser beam 120 emitted from the distal end 204 is incident on the target 108. In such an orientation, the reflected light 122 is reflected from the target 108 (e.g., as a result of the interrogation laser beam 120 being incident on the target 108). The reflected light 122 is received at the distal end 204 of the optical fiber 104 and transmitted to the proximal end 202 where it is coupled to the surgical laser console 102.

In such a configuration, where therapeutic laser beam 118 is generated by the therapeutic laser source 112, it will also be emitted from the distal end 204 and be incident on the target 108 and the target can be treated (e.g., fragmented, dusted, destroyed, etc.)

Turning to FIG. 2B, the optical fiber 104 is depicted targeting the non-target object 110. Said differently, the distal end 204 is pointed at the non-target object 110 such that the interrogation laser beam 120 emitted from the distal end 204 is incident on the non-target object 110. In such an orientation, light is reflected from the non-target object 110 (e.g., as a result of the interrogation laser beam 120 being incident on the non-target object 110). This light is depicted as non-target reflected light 208, which like the reflected light 122 is received at the distal end 204 of the optical fiber 104 and transmitted to the proximal end 202 where it is coupled to the surgical laser console 102.

In such a configuration, where therapeutic laser beam 118 is generated by the therapeutic laser source 112, it will also be emitted from the distal end 204 and be incident on the non-target object 110. It is to be appreciated that in such a configuration, it may not be desirable to generate therapeutic laser beam 118 so as not to damage the non-target object 110.

Turning to FIG. 2C, the optical fiber 104 is depicted targeting an edge 210 of the target 108. Said differently, the distal end 204 is pointed at the edge 210 of the target 108 such that the interrogation laser beam 120 emitted from the distal end 204 is incident on the edge 210 of the target 108. In such an orientation, part of the interrogation laser beam 120 will be incident on the edge 210 of the target 108 and the other part will be incident on the non-target object 110. As such, non-target reflected light 208 will be reflected from the portion of the interrogation laser beam 120 incident on the non-target object 110 while edge reflected light 212 will be reflected from the portion of the interrogation laser beam 120 incident on the edge 210 of the target 108.

Both the non-target reflected light 208 and the edge reflected light 212 can be received at the distal end 204 of the optical fiber 104 and be transmitted to the proximal end 202 where it is coupled to the surgical laser console 102.

In such a configuration, where therapeutic laser beam 118 is generated by the therapeutic laser source 112, it will also be emitted from the distal end 204 and a portion will be incident on the edge 210 of the target 108 while the other portion will be incident on the non-target object 110. It is to be appreciated that in such a configuration, it may not be desirable to generate therapeutic laser beam 118 so as not to damage the non-target object 110.

Returning to FIG. 1, as described above, the processing unit 124 can execute instructions 128 to cause the computing sub-system 116 to determine whether the target 108, the non-target object 110, or the edge, is being targeted by the optical fiber 104 based on the reflected light 122. For example, responsive to incidence of interrogation laser beam 120 on the target 108, the non-target object 110, or the edge 210, light having different fluorescence will be reflected. For example, the target reflected light 206 will have a different fluorescence than the non-target reflected light 208 and the edge reflected light 212. The processing unit 124 can execute instructions 128 to determine whether the target 108, the non-target object 110, or the edge 210 are being targeted based on the fluorescence of the reflected light (e.g., target reflected light 206, non-target reflected light 208, edge reflected light 212, or the like).

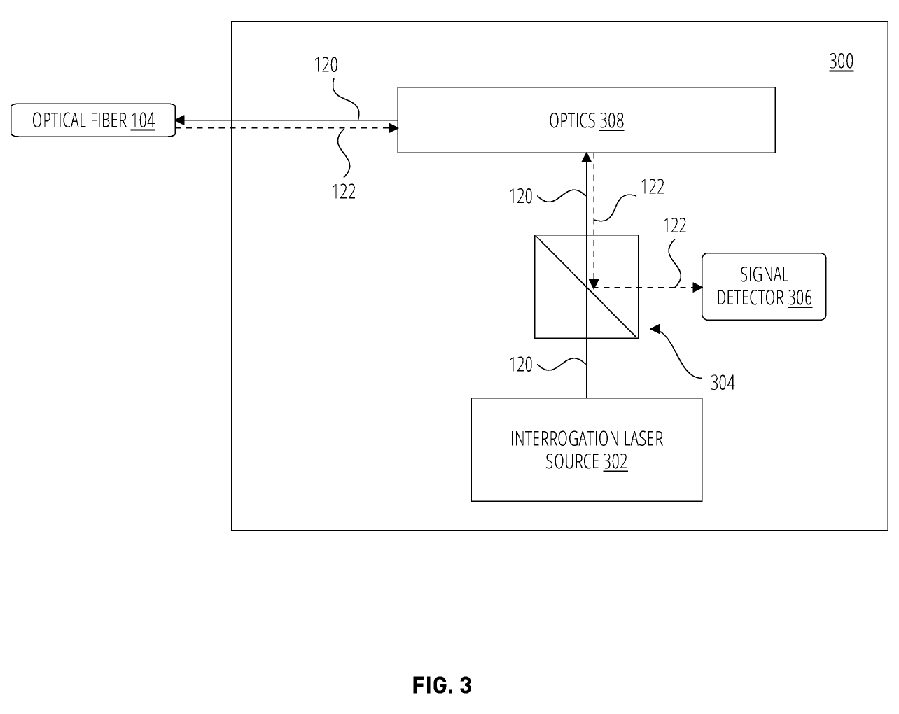

FIG. 3 illustrates an example interrogation sub-system 300, which can be implemented as interrogation sub-system 114 of surgical laser system 100 of FIG. 1. The interrogation sub-system 300 can include an interrogation laser source 302, a beam splitter 304, a signal detector 306, and optics 308. The interrogation laser source 302 can be arranged to generate interrogation laser beam 120 via any of a variety of lasing mechanisms. For example, interrogation laser source 302 can comprise diode lasers. The interrogation laser beam 120 can be directed to beam splitter 304, which directs the interrogation laser beam 120 to optics 308.

The optics 308 can comprise any of a variety of optical component arranged to condition and direct the interrogation laser beam 120 from beam splitter 304 to optical fiber 104 and direct the reflected light 122 from optical fiber 104 to beam splitter 304. For example, the optics 308 can include polarizers, beam combiners, collimators, circulators, lenses, etc.

The beam splitter 304 may further be configured to direct the reflected light 122 to the signal detector 306. The beam splitter 304 may include any of a variety of optical components used to direct incident light from the interrogation laser source 302 to the optics 308 and to direct incident light from the optics 308 to the signal detector 306.

The signal detector 306 can be any of a variety of light detectors configured to measure an intensity and/or wavelength of the reflected light 122 and output electrical signals based on the measure intensity and/or wavelength. For example, signal detector 306 can be a photomultiplier tube, a photodiode, or avalanche photodiodes. Electrical signals output from the signal detector 306 can be communicated to a computing device (e.g., the computing sub-system 116, or the like) and used to determine whether the target 108, non-target object 110, or edge 210 are being targeted by the distal end 204 of the optical fiber 104.

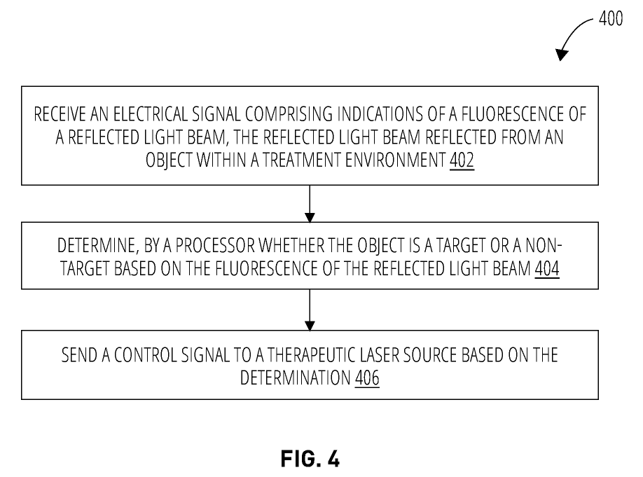

FIG. 4 illustrates a method 400 for distinguishing between a target and a non-target, according to at least one embodiment of the present disclosure. The method 400 can be implemented by a laser emitting medical device, such as, for example, the surgical laser system 100 of FIG. 1. The method 400 will be described with reference to the surgical laser system 100 as well as to the interrogation sub-system 300 for clarity of presentation. However, it is noted that method 400 could also be implemented by a laser emitting medical device different from the surgical laser system 100 of FIG. 1 without departing from the scope of the disclosure.

The method 400 can begin at block 402. At block 402 “receive an electrical signal comprising indications of a fluorescence of a reflected light beam, the reflected light beam reflected from an object within a treatment environment” an electrical signal can be received, where the electrical signal comprises indications of fluorescence of light reflected from an object. For example, processing unit 124 can execute instructions 128 to receive electrical signals from signal detector 306 comprising indications of a fluorescence of the reflected light 122.

Continuing to block 404 “determine, by a processor whether the object is a target, or a non-target based on the fluorescence of the reflected light beam” a determination of whether the object is a target, or a non-target can be made. For example, processing unit 124 can execute instructions 128 to determine whether the distal end 204 of the optical fiber 104 is pointed at (e.g., targeting, or the like) the target 108 or the non-target object 110 based on the electrical signals received at block 402 (e.g., the fluorescence of the reflected light 122).

Continuing to block 406 “send a control signal to a therapeutic laser source based on the determination” a control signal, based on the determination, can be sent to a therapeutic laser source. For example, processing unit 124 can execute instructions 128 to cause computing sub-system 116 to send a control signal to the therapeutic laser source 112 where the control signal is based on the determination of whether the object is the target 108 or the non-target object 110. For example, based on a determination that the object is the target 108, the computing sub-system 116 can send a control signal to the therapeutic laser source 112 to cause the therapeutic laser source 112 to generate the therapeutic laser beam 118 while the computing sub-system 116 can send a control signal to the therapeutic laser source 112 to cause the therapeutic laser source 112 to not generate the therapeutic laser beam 118 based on a determination that the object is the non-target object 110.

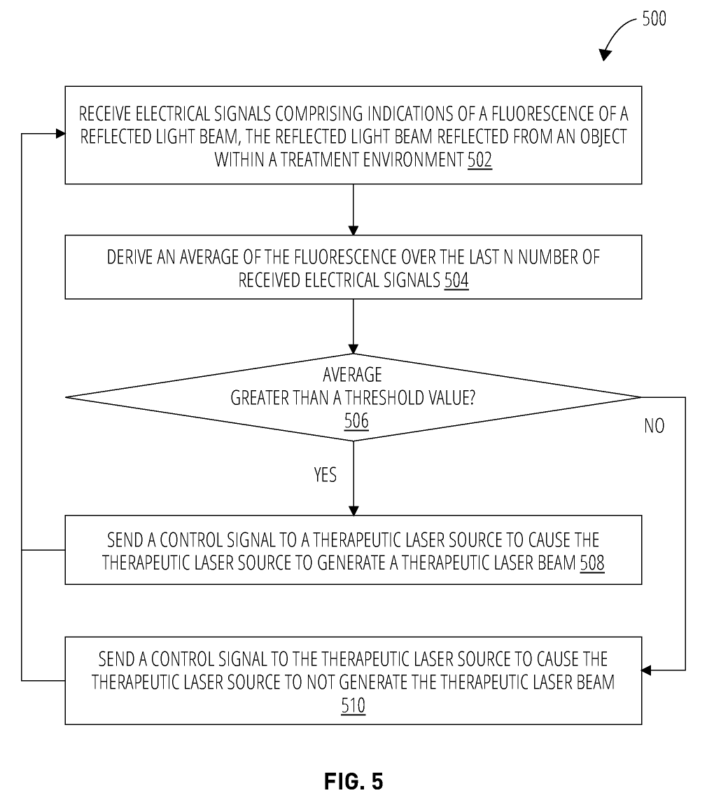

FIG. 5 illustrates a method 500 for distinguishing between a target and a non-target, according to at least one embodiment of the present disclosure. The method 500 can be implemented by a laser emitting medical device, such as, for example, the surgical laser system 100 of FIG. 1. The method 500 will be described with reference to the surgical laser system 100 as well as to the interrogation sub-system 300 for clarity of presentation. However, it is noted that method 500 could also be implemented by a laser emitting medical device different from the surgical laser system 100 of FIG. 1 without departing from the scope of the disclosure.

The method 500 can begin at block 502. At block 502 “receive electrical signals comprising indications of a fluorescence of a reflected light beam, the reflected light beam reflected from an object within a treatment environment” electrical signals can be received, where the electrical signal comprises indications of fluorescence of light reflected from an object. For example, processing unit 124 can execute instructions 128 to receive electrical signals from signal detector 306 comprising indications of a fluorescence of the reflected light 122 over a time period. Said differently, processing unit 124 can execute instructions 128 to receive several (e.g., 4, 6, 8, 10, 12, 16, 24, 32, 64, 128, between 4 and 128, etc.) samples or outputs from signal detector 306.

Continuing to block 504 “derive an average of the fluorescence over the last N number of received electrical signals” an average of the fluorescence of over the last N (e.g., 6, 64, etc.) received electrical signals can be derived. For example, processing unit 124 can execute instructions 128 to derive an average of the fluorescence of the last N received electrical signals.

Continuing to decision block 506 “average greater than a threshold value?” a determination can be made as to whether the average is greater than a threshold value. For example, processing unit 124 can execute instructions 128 to determine whether the average of the fluorescence over the last N samples is greater than a threshold value. With some embodiments, the threshold can be a minimum fluorescence associated with the target 108, or the like.

From decision block 506, the method 500 can continue to either block 508 or block 510. For example, method 500 can continue from decision block 506 to block 508 based on a determination that the average fluorescence is greater than (or greater than or equal to) the threshold while method 500 can continue from decision block 506 to block 510 based on a determination that the average fluorescence is not greater than the threshold.

At block 508 “send a control signal to a therapeutic laser source to cause the therapeutic laser source to generate a therapeutic laser beam” a control signal can be sent to a therapeutic laser source to cause the therapeutic laser source to generate a therapeutic laser beam. For example, processing unit 124 can execute instructions 128 to cause computing sub-system 116 to send a control signal to the therapeutic laser source 112 to cause the therapeutic laser source 112 to generate the therapeutic laser beam 118.

At block 508 “send a control signal to the therapeutic laser source to cause the therapeutic laser source to not generate the therapeutic laser beam” a control signal can be sent to the therapeutic laser source to cause the therapeutic laser source to not generate the therapeutic laser beam. For example, processing unit 124 can execute instructions 128 to cause computing sub-system 116 to send a control signal to the therapeutic laser source 112 to cause the therapeutic laser source 112 to not generate the therapeutic laser beam 118.

From block 508 and block 510, the method 500 can return to block 502 where more samples can be received and another determination as to whether the target 108 or the non-target object 110 is targeted can be made.

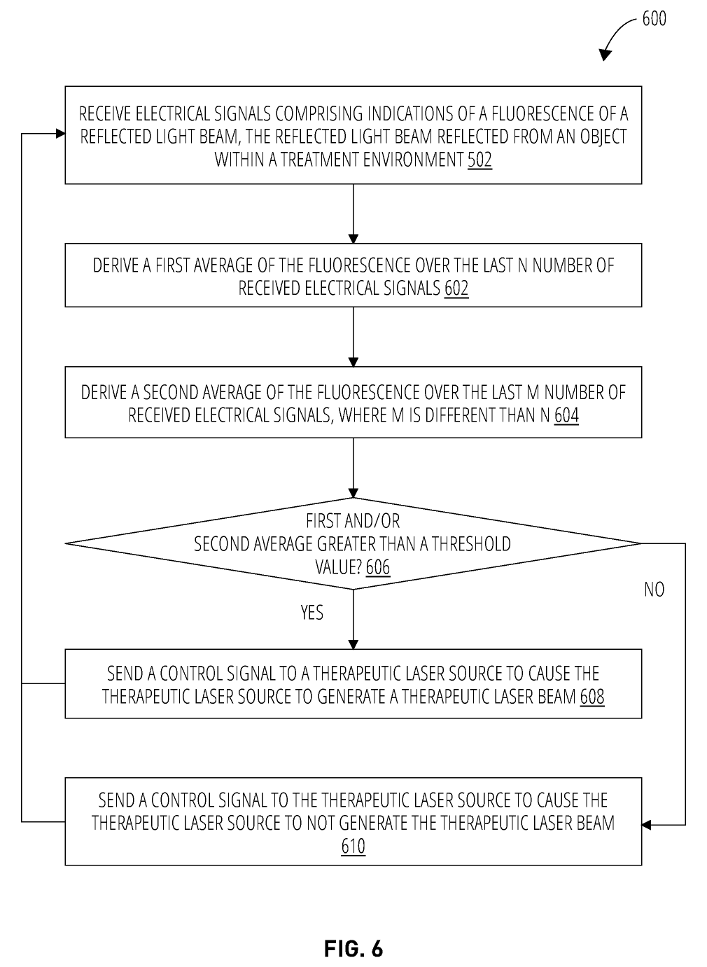

FIG. 6 illustrates a method 600 for distinguishing between a target and a non-target, according to at least one embodiment of the present disclosure. The method 600 can be implemented by a laser emitting medical device, such as, for example, the surgical laser system 100 of FIG. 1. The method 600 will be described with reference to the surgical laser system 100 as well as to the interrogation sub-system 300 for clarity of presentation. However, it is noted that method 600 could also be implemented by a laser emitting medical device different from the surgical laser system 100 of FIG. 1 without departing from the scope of the disclosure.

The method 600 can begin at block 502 and continue to block 602. At block 602 “derive a first average of the fluorescence over the last N number of received electrical signals” an average of the fluorescence of over the last N (e.g., 6, 64, etc.) received electrical signals can be derived. For example, processing unit 124 can execute instructions 128 to derive an average of the fluorescence of the last N received electrical signals. Continuing to block 604 “derive a second average of the fluorescence over the last M number of received electrical signals, where M is different than N” an average of the fluorescence of over the last M (e.g., 6, 64, etc.) received electrical signals can be derived. For example, processing unit 124 can execute instructions 128 to derive an average of the fluorescence of the last M received electrical signals. As a specific example, N can be 64 samples while M can be 6 samples. With some embodiments, N is less than 32 and M is greater than 32. In some embodiments, N is between 3 and 31 and M is between greater than 32 and 96.

Continuing to decision block 606 “first and/or second average greater than a threshold value?” a determination can be made as to whether the first and/or the second average is greater than a threshold value. For example, processing unit 124 can execute instructions 128 to determine whether the first average (e.g., average over N samples) and/or the second average (e.g., average over M samples) is greater than a threshold value. As a specific example, processing unit 124 can execute instructions 128 to determine whether the average fluorescence over 64 samples and the average fluorescence over 6 samples is greater than the threshold.

From decision block 606, the method 600 can continue to either block 608 or block 610. For example, method 600 can continue from decision block 606 to block 608 based on a determination that the first and/or the second average fluorescence is greater than (or greater than or equal to) the threshold while method 600 can continue from decision block 606 to block 610 based on a determination that the first and/or the second average fluorescence is not greater than the threshold.

At block 608 “send a control signal to a therapeutic laser source to cause the therapeutic laser source to generate a therapeutic laser beam” a control signal can be sent to a therapeutic laser source to cause the therapeutic laser source to generate a therapeutic laser beam. For example, processing unit 124 can execute instructions 128 to cause computing sub-system 116 to send a control signal to the therapeutic laser source 112 to cause the therapeutic laser source 112 to generate the therapeutic laser beam 118.

At block 608 “send a control signal to the therapeutic laser source to cause the therapeutic laser source to not generate the therapeutic laser beam” a control signal can be sent to the therapeutic laser source to cause the therapeutic laser source to not generate the therapeutic laser beam. For example, processing unit 124 can execute instructions 128 to cause computing sub-system 116 to send a control signal to the therapeutic laser source 112 to cause the therapeutic laser source 112 to not generate the therapeutic laser beam 118.

From block 608 and block 610, the method 600 can return to block 604 where more samples can be received and another determination as to whether the target 108 or the non-target object 110 is targeted can be made.

It is noted that when the distal end 204 of the optical fiber 104 is positioned in front of the edge 210 of a urinary stone (e.g., the target 108), the fluorescence of the reflected light 122 will drop significantly compared to when the distal end 204 of the optical fiber 104 is positioned in front of, or centered on, the urinary stone (e.g., the target 108). Accordingly, the method 600 provides to determine whether it is safe to enable therapeutic lasing (e.g., generation of the therapeutic laser beam 118, or the like) based on derived changes in the fluorescence of reflected light responsive to incidence of the interrogation laser beam on an object (e.g., the target 108, the non-targeted object 110, or the like). As outlined above, the method 600 provides to derive two averages of the fluorescence over different time periods (e.g., the last 6 samples and the last 64 samples, or the like). As such, the method 600 provides to enable lasing where the averages over both time periods are greater than or equal to the threshold, thereby monitoring both slow and fast changes to the fluorescence ensure that lasing is only enabled when a urinary stone (e.g., the target 108, or the like) is targeted, or in front of the distal end 204 of the optical fiber 104.

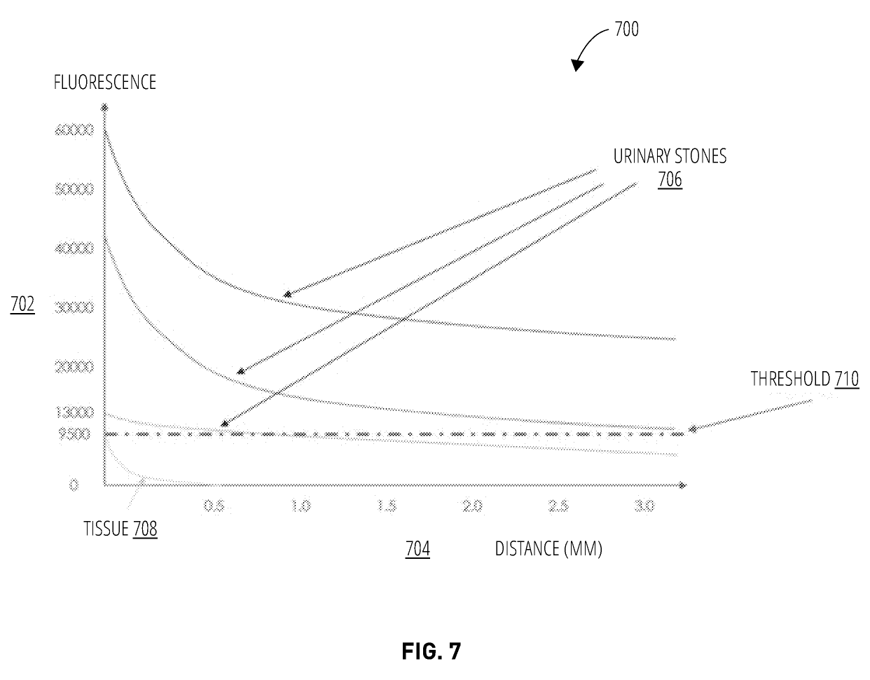

FIG. 7 illustrates a plot 700 showing example fluorescence that can be measured from urinary stones and tissue as part of a lithotripsy procedure as contemplated herein. For example, the plot 700 depicts fluorescence of reflected light on the y axis 702 against distance (measured in millimeters (mm)) of the object from the distal end 204 of the optical fiber on the x axis 704. For example, fluorescence of reflected light for a number of urinary stones 706 and tissue 708 in plotted in plot 700. In general, the intensity of the fluorescence is plotted on the y axis 702.

As discussed above the average of the fluorescence over a time period (e.g., the last 6 samples, the last 64 samples, or the like) can be derived and where the average is greater than or equal to a threshold therapeutic lasing can be enabled. With some embodiments, the threshold 710 can be based on the fluorescence of the tissue 708 versus urinary stones 706. For example, plot 700 depicts the threshold 710 at just above the fluorescence of the tissue 708.

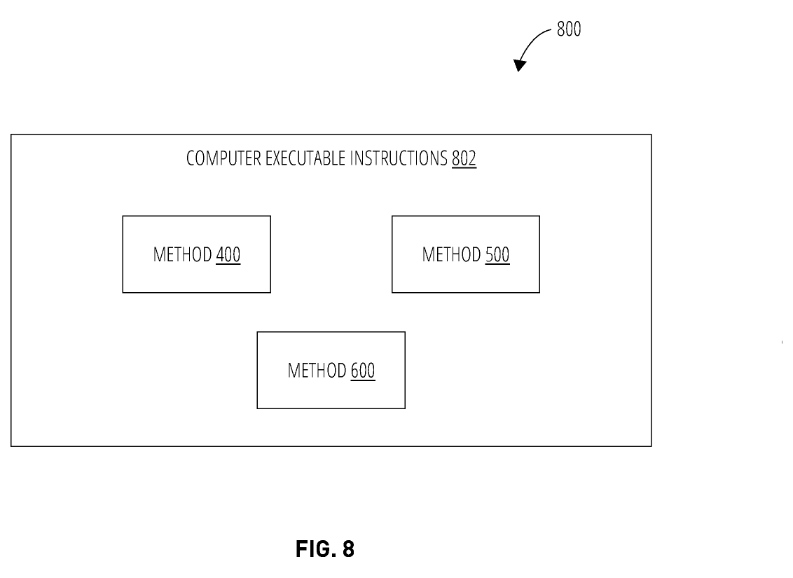

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, computer-readable storage medium 800 may store computer executable instructions 802 with which circuitry (e.g., computing sub-system 116, processing unit 124, or the like) can execute. For example, computer executable instructions 802 can include instructions to implement operations described with respect to method 400, method 500, and/or method 600. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

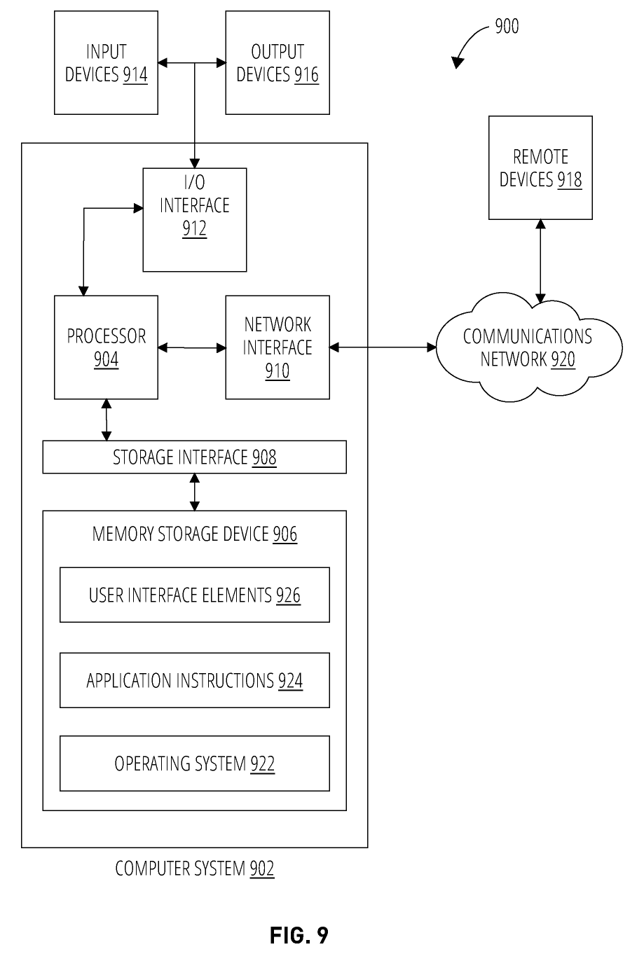

FIG. 9 is a block diagram of a computing environment 900 including a computer system 902 for implementing embodiments consistent with the present disclosure. In some embodiments, the computing environment 900, or portion thereof (e.g., the computer system 902) may comprise or be comprised in a laser system (e.g., the computing sub-system 116 of the surgical laser system 100 can embody portions of the computing environment 900). Accordingly, in various embodiments, computer system 902 may distinguish between a targeted and non-target object (e.g., between a urinary stone and tissue) during a surgical laser procedure (e.g., laser lithotripsy, or the like).

The computer system 902 may include a central processing unit (“CPU” or “processor”) 904. The processor 904 may include at least one data processor for executing instructions and/or program components for executing user or system-generated processes. A user may include a person, a person using a device such as those included in this disclosure, or another device. The processor 904 may include specialized processing units such as integrated system (bus) controllers, memory management control units, floating point units, graphics processing units, neural processing units, digital signal processing units, etc. The processor 904 may be disposed in communication with input devices 914 and output devices 916 via I/O interface 912. The I/O interface 912 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n /b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 912, computer system 902 may communicate with input devices 914 and output devices 916. In some embodiments, the processor 904 may be disposed in communication with a communications network 920 via a network interface 910. In various embodiments, the communications network 920 may be utilized to communicate with a remote memory storage device 906, such as for accessing look-up tables, performing updates, or utilizing external resources. The network interface 910 may communicate with the communications network 920. The network interface 910 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 920 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 920 may include a variety of network devices, including routers, bridges, servers, computing devices, storage devices, etcetera. In some embodiments, the processor 904 may be disposed in communication with a memory storage device 906 via a storage interface 908. The storage interface 908 may connect to memory storage device 906 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 906 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 906 may store a collection of program or database components, including, without limitation, an operating system 922, application instructions 924, and user interface elements 926. In various embodiments, the operating system 922 may facilitate resource management and operation of the computer system 902. Examples of operating systems include, without limitation, APPLE^{®} MACINTOSH^{®} OS X^{®}, UNIX^{®}, UNIX-like system distributions (E.G., BERKELEY SOFTWARE DISTRIBUTION^{®} (BSD), FreeBSD^{®}, NetBSD^{®}, OpenBSD^{®}, etc.), LINUX^{®} DISTRIBUTIONS (E.G., RED HAT^{®}, UBUNTU^{®}, KUBUNTU^{®}, etc.), IBM^{®}OS/2^{®}, MICROSOFT^{®} WINDOWS^{®} (XP^{®}, VISTA^{®}/7/8, 10 etc.), APPLE^{®} IOS^{®}, GOOGLE^{TM} ANDROID^{TM}, BLACKBERRY^{®} OS, or the like.

The application instructions 924 may include instructions that when executed by the processor 904 cause the processor 904 to perform one or more techniques, steps, procedures, and/or methods described herein, such as to distinguish between the target 108 and the non-target object 110 based on reflected light 122 or rather based on signals received from signal detector 306.

The user interface elements 926 may facilitate display, execution, interaction, manipulation, or operation of program components through textual or graphical facilities. For example, user interfaces may provide computer interaction interface elements on a display system operatively connected to the computer system 902, such as cursors, icons, checkboxes, menus, scrollers, windows, widgets, etcetera. The user interface elements 926 may be employed by application instructions 924 and/or operating system 922 to provide, for example, a user interface with which a user can interact with computer system 902. In some embodiments, the user interface elements 926 may be displayed on a display.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A surgical laser system, comprising:
a therapeutic laser source configured to generate a therapeutic laser beam;
an interrogation sub-system comprising:
an interrogation laser source configured to generate an interrogation laser beam,
a light detector, and
a beam splitter;
optics configured to condition the therapeutic laser beam and the interrogation laser beam and couple the therapeutic laser beam and the interrogation laser beam to an optical fiber;
a processor coupled to the light detector; and
memory coupled to the processor, the memory comprising instructions that when executed cause the processor to:
receive, from the light detector, electrical signals comprising indications of a fluorescence of reflected light,
derive an average of the fluorescence over the last N samples of the electrical signals,
determine whether the average of the fluorescence over the last N samples is greater than or greater than or equal to a threshold value, and
send, responsive to the determination, a control signal to the therapeutic laser source,
wherein the reflected light is light reflected from an object responsive to incidence of the interrogation laser beam on the object.

2. The surgical laser system of claim 1, the instructions when executed by the processor further cause the processor to:
send a first control signal to the therapeutic laser source based on a determination that the average of the fluorescence over the last N samples is greater than or greater than or equal to the threshold value; or
send a second control signal to the therapeutic laser source based on a determination that the average of the fluorescence over the last N samples is not greater than or not greater than or equal to the threshold value,
wherein the first control signal causes the therapeutic laser source to generate a therapeutic laser beam, and
wherein the second control signal causes the therapeutic laser source to not generate the therapeutic laser beam.

3. The surgical laser system of claim 1, the instructions when executed by the processor further cause the processor to:
derive an average of the fluorescence over the last M samples of the electrical signals, where M is different than N; and
determine whether the average of the fluorescence over the last M samples is greater than the threshold value,
wherein sending the control signal to the therapeutic laser source is responsive to both determinations.

4. The surgical laser system of claim 3, the instructions when executed by the processor further cause the processor to:
send a first control signal to the therapeutic laser source based on a determination that the average of the fluorescence over the last N samples and the average fluorescence over the last M samples is greater than or greater than or equal to the threshold value; or
send a second control signal to the therapeutic laser source based on a determination that the average of the fluorescence over the last N samples or the average fluorescence over the last M samples is not greater than or not greater than or equal to the threshold value,
wherein the first control signal causes the therapeutic laser source to generate a therapeutic laser beam, and
wherein the second control signal causes the therapeutic laser source to not generate the therapeutic laser beam.

5. The surgical laser system of claim 3, wherein N is 6 and M is 64.

6. The surgical laser system of claim 1, wherein the object is a urinary stone.

7. At least one machine readable storage devices storing instructions that when executed by a processor of a surgical laser system cause the surgical laser system to:
receive, from a light detector, electrical signals comprising indications of a fluorescence of reflected light,
derive an average of the fluorescence over the last N samples of the electrical signals,
determine whether the average of the fluorescence over the last N samples is greater than or greater than or equal to a threshold value, and
send, responsive to the determination, a control signal to a therapeutic laser source of the surgical laser system,
wherein the reflected light is light reflected from an object responsive to incidence of an interrogation laser beam, generated by the surgical laser system, on the object.

8. The at least one machine readable storage devices of claim 7, the instructions when executed by the processor further cause the surgical laser system to:
send a first control signal to the therapeutic laser source based on a determination that the average of the fluorescence over the last N samples is greater than or greater than or equal to the threshold value; or
send a second control signal to the therapeutic laser source based on a determination that the average of the fluorescence over the last N samples is not greater than or not greater than or equal to the threshold value,
wherein the first control signal causes the therapeutic laser source to generate a therapeutic laser beam, and
wherein the second control signal causes the therapeutic laser source to not generate the therapeutic laser beam.

9. The at least one machine readable storage devices of claim 7, the instructions when executed by the processor further cause the surgical laser system to:
derive an average of the fluorescence over the last M samples of the electrical signals, where M is different than N; and
determine whether the average of the fluorescence over the last M samples is greater than the threshold value,
wherein sending the control signal to the therapeutic laser source is responsive to both determinations.

10. The at least one machine readable storage devices of claim 9, the instructions when executed by the processor further cause the surgical laser system to:
send a first control signal to the therapeutic laser source based on a determination that the average of the fluorescence over the last N samples and the average fluorescence over the last M samples is greater than or greater than or equal to the threshold value; or
send a second control signal to the therapeutic laser source based on a determination that the average of the fluorescence over the last N samples or the average fluorescence over the last M samples is not greater than or not greater than or equal to the threshold value,
wherein the first control signal causes the therapeutic laser source to generate a therapeutic laser beam, and
wherein the second control signal causes the therapeutic laser source to not generate the therapeutic laser beam.

11. The at least one machine readable storage devices of claim 10, wherein N is 6 and M is 64.

12. The at least one machine readable storage devices of claim 10, wherein the object is a urinary stone.

13. A computer implemented method, comprising:
receiving, at a processor, electrical signals generated by a light detector, the electrical signals comprising indications of a fluorescence of reflected light received at the light detector;
deriving an average of the fluorescence over the last N samples of the electrical signals;
determining whether the average of the fluorescence over the last N samples is greater than or greater than or equal to a threshold value; and
sending, responsive to the determination, a control signal to a therapeutic laser source.

14. The computer implemented method of claim 13, wherein sending the control signal to the therapeutic laser source comprises:
sending a first control signal to the therapeutic laser source based on a determination that the average of the fluorescence over the last N samples is greater than or greater than or equal to the threshold value; or
sending a second control signal to the therapeutic laser source based on a determination that the average of the fluorescence over the last N samples is not greater than or not greater than or equal to the threshold value,
wherein the first control signal causes the therapeutic laser source to generate a therapeutic laser beam, and
wherein the second control signal causes the therapeutic laser source to not generate the therapeutic laser beam.

15. The computer implemented method of claim 14, further comprising:
deriving an average of the fluorescence over the last M samples of the electrical signals, where M is different than N; and
determining whether the average of the fluorescence over the last M samples is greater than the threshold value,
wherein sending the control signal to the therapeutic laser source is responsive to both determinations.

16. The computer implemented method of claim 15, wherein sending the control signal to the therapeutic laser source comprises:
sending a first control signal to the therapeutic laser source based on a determination that the average of the fluorescence over the last N samples and the average fluorescence over the last M samples is greater than or greater than or equal to the threshold value; or
sending a second control signal to the therapeutic laser source based on a determination that the average of the fluorescence over the last N samples or the average fluorescence over the last M samples is not greater than or not greater than or equal to the threshold value,
wherein the first control signal causes the therapeutic laser source to generate a therapeutic laser beam, and
wherein the second control signal causes the therapeutic laser source to not generate the therapeutic laser beam.

17. The computer implemented method of claim 15, comprising:
generating, by an interrogation sub-system during a surgical laser procedure, an interrogation laser beam; and
coupling the interrogation laser beam to an optical fiber,
wherein the optical fiber is disposed in a treatment environment, and
wherein the reflected light is light reflected from an object in the treatment environment responsive to incidence of the interrogation laser beam on the object.

18. The computer implemented method of claim 17, wherein N is less than 32 and M is greater than 32.

19. The computer implemented method of claim 17, wherein N is between 3 and 31 and M is between 32 and 96.

20. The computer implemented method of claim 13, wherein the object is a urinary stone.
